(19) United States
(12) Patent Application Publication
(10) Pub. No.: US 2007/0092634 A1
(43) Pub. Date: Apr. 26, 2007

(54) Method and device for decorating fingernails with multicolored designs

(76) Inventor: Xingzhu Zhang, Zhengzhou City (CN)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 10/565,721

(22) PCT Filed: Apr. 29, 2005
(86) PCT No.: PCT/CN05/00599

§ 371 Date: Jan. 24, 2006

(30) Foreign Application Priority Data
May 13, 2004 (CN) 200410037923.0

(51) Int. Cl. A61B 5/117; B05D 5/00; B05C 11/00

(52) U.S. Cl. 427/1; 427/256; 118/713; 118/686

(57) ABSTRACT

The present invention relates to a body painting method mainly used in nail beautifying for simultaneously creating a plurality of patterns, and a specialized computer-controlled body-painting machine for realizing the method. In use, a plurality of target body parts to be painted are simultaneously placed onto the clamp of the body-painting machine after necessary preprocesses have been preformed to the target body parts; then a plurality of pictures for said body parts are simultaneously opened with a computer and the body parts are displayed on the screen of the computer by the camera, the pictures will be printed as one pattern simultaneously after the sizes and printing locations of these pictures have been adjusted, resulting in each of the pictures being printed at its corresponding printing location; the body-painting machine is operated by the computer, and its printing head is connected to a transverse moving mechanism and a longitudinal moving mechanism; the camera head is fixedly mounted above the printing head; the clamp can be inserted into the space between the upper casing and the base of the machine body. According to present invention, a plurality of body-painting patterns can be printed at one time, thereby the painting process is greatly simplified and the painting time is significantly reduced.

### FIELD OF THE INVENTION

The present invention relates to a body painting method for simultaneously creating a plurality of patterns, which mainly used in beautifying the nails, and to a specialized computer-controlled body-painting machine for implementing the method.

### BACKGROUND OF THE INVENTION

The conventional method for body painting such as nail beautifying and the like is as follows. Pluralities of pictures are stored in a computer. In operation, a selected picture is firstly opened by the computer, then a nail or some other part of a human body to be painted is held on the clamp of the body-painting machine, and the relative position of said nail or said other part of a human body to be painted is determined by a camera and displayed on the screen of the computer, after that, the size and the relative position of the picture are then adjusted so that the size of the pattern is equal to or larger than the size of the body part to be painted and the printing location is positioned right at said body part. A printing device is then started which prints the picture onto said part so as to form a body painting pattern. A preprocess such as applying base oil to the body part to be painted can be performed if needed, and those parts of the body-painting pattern outside the target body part such as a nail may be erased after printing, and the body painting may then be provided with a protection coating, whereby the whole body painting of a single pattern is finished. The drawback of such a method for body painting is that only one pattern can be printed at a time, in the case that a plurality of patterns are needed to be printed onto nails or the like, they can only be printed one by one with this method, resulting a very inconvenient and time consuming operation.

### SUMMARY OF THE INVENTION

In order to overcome the disadvantage of above-mentioned conventional method, in which only one painting pattern can be printed at a time, the present invention provides a body painting method for simultaneously creating a plurality of patterns, and a specialized computer-controlled body-painting machine for realizing the method. The method and machine according to the present invention are capable of printing a plurality of body painting patterns simultaneously, which not only simplify the process, but also save time.

The technical solution for achieving the above object is a body painting method for simultaneously creating a plurality of patterns, which employs a specialized computer-controlled body-painting machine to print the body-painting patterns, comprises the following basic steps:

(1) Opening a plurality of pictures used for body paintings simultaneously in a computer;

(2) Placing objects having a plurality of parts to be painted onto a corresponding clamp, and holding each part within the printing area of a printing device by means of the clamp;

(3) Determining the location of each part by a camera and displaying them on a display of the computer, and adjusting the size and printing location of each opened picture through the computer so that the size of each picture is equal to or larger than the size of its respective body part to be painted while the printing location of each picture is positioned right at said respective body part;

(4) Starting the printing device by the computer so that all pictures are printed as one pattern by the printing device, while each picture is printed right onto its respective body part.

The technical solution according to the present invention also includes a computer-controlled body-painting machine for simultaneously creating a plurality of body painting patterns, which comprises a computer, a printing device, a camera, a longitudinal moving mechanism, a transverse moving mechanism, a clamp and a machine body; the computer is electrically connected to the printing device and the camera, the machine body mainly comprises an upper casing and a base; the upper casing is located above the base, the printing device mainly comprises a printing head which forms the moving member of the transverse moving mechanism or is fixedly mounted on the moving member of the transverse moving mechanism, the transverse move mechanism comprises a fixed member; a moving member and a moving member driving means, the fixed member of which is connected to the moving member of the longitudinal moving mechanism, the longitudinal moving mechanism comprises a fixed member, a moving member and a moving member driving means, the fixed member of which is connected to the upper part of the inner space of the upper casing, the camera mainly comprises a camera head which is positioned above the printing head and is arranged in such way that the shooting direction faces in the downward direction, and the shooting area covers the printing area of the printing head; there is provided with a space between the upper casing and the base to receive the clamp, the front side of the space (facing the operator) is opened, the clamp is movably disposed within the space for receiving the same therein between the upper casing and the base, the position of the painting body part determined by which is located in the printing area.

A plurality of body-painting patterns can be printed as one pattern simultaneously, according to the adjusted printing location, onto each corresponding painting location. That is to say, only one printing operation is needed for forming said pluralities of body-painting patterns, thereby greatly simplifies the painting process and saves time.

### BRIEF DESCRIPTION OF THE DRAWINGS

FIG. 1 is a schematic front view of the structure of the body-painting machine according to the present invention;

FIG. 2 is a schematic side view of the structure of the body-painting machine according to the present invention.

### DETAILED DESCRIPTION OF PREFERRED EMBODIMENTS OF THE INVENTION

The body painting method according to the present invention mainly comprises the following basic steps:

(1) A plurality of pictures used for body painting are opened simultaneously on a computer screen;

(2) Objects having a plurality of parts to be painted are placed onto corresponding clamps, and each part to be painted is held within the printing area of the printing device by the clamps;

(3) The location of each part is determined and displayed on the screen by a camera, and the size and printing location of each opened picture are adjusted by the computer so that the size of each picture is equal to or larger than the size of the respective body part to be painted while the printing location is positioned right at said respective body part;

(4) The printing device is started by the computer and all pictures are printed as one pattern so that each picture is printed right onto its corresponding part.

A preprocess to the part to be painted can be performed before printing according to the properties of different parts of human body so that the part may meet the requirements of printing and human body protection. A post-process can also be performed upon completion of printing so that the printed patterns may meet the requirements of aesthetics and can be protected.

Taking fingernails and toenails as examples, before printing, the surfaces of the nails can be applied with calcium-enriched base oil and then with an ink absorbing layer, or the grease layer on the nails may be removed with nail oil remover or alcohol, after printing, a protection solution can be applied to the surfaces of the nails so as to form a protection layer.

Body-painting patterns can be arbitrarily selected from the pictures stored in the computer, the selected picture can be the same or different. Because the shape of a stored picture is generally rectangular and cannot fit completely with the shape of a particular body part to be painted (the surface of a nail, for example), therefore, modification to the pattern is needed. The modification of a pattern can be achieved in the following two ways or any other suitable ways:

The first way concerns having the edges of a selected picture trimmed so that the picture can fit completely with a body part to be painted, the detailed process of which is: in said step (3), a static photo illustrating the location of each part to be painted is taken by the camera and these static photos are displayed on the screen as the bottom layer of the pictures adjusting working area; then each of the opened pictures is adjusted on the screen to the position corresponding to the part to be painted, and each picture is presented semitransparent so that the profile of the nail beneath the picture can be seen clearly, and the size of each picture is then adjusted so that it is slightly larger than that of the painting part, and then a profile line of the painting part is drawn and the pixels of the picture outside the profile is removed so that the edges of the picture overlap with the edges of the nail.

The second way concerns having the surplus parts of the printed pattern erased after printing, and only the parts of the printed pattern on the painting part is remained. The detailed process of which is: In said step (3), the size of each picture is adjusted so that it is equal to or slightly larger than that of the body part, and those parts of the pattern beyond the target painting part are then erased using suitable detergents after printing. A coating layer that can be readily washed off can be firstly applied to the surrounding of the painting part in the preprocess procedure so as to facilitate the erase of the printing pattern.

There are two convenient embodiments for nail paintings:

Embodiment 1 (nail beautifying painting patterns are created on all ten nails of a pair of hands):

(a) Applying an ink absorbing layer to the surfaces of the ten nails to be beautified, or removing the grease layer on the nails using nail oil remover or alcohol;

(b) Selecting five pictures by means of a drawing software in the computer and opening them simultaneously;

(c) Stretching out the four fingers of one hand except the thumb and the thumb of the other hand, and arranging them into a row, them placing them in a finger holder used as the clamp with the surfaces of the fingers facing upward, and holding the surfaces of the fingers lined-up within the printing area by the finger holder;

(d) Determining the position of each finger by the camera and displaying it on the screen;

(e) Adjusting the position of each picture with respect to its target nail surface by means of operating the computer so that they overlap with each other, and adjusting the size of each picture so that it is slightly larger than the corresponding nail surface, then trimming the edges of each picture by the edge modifying tools of the software so that its edges overlap with the edges of the nail surface;

(f) Issuing the command for printing by means of the computer to the printing device such that the five pictures are simultaneously printed, thereby five patterns are formed on each respective surface of the five nails;

(g) Repeating the steps (b)-(f) in order to print painting patterns on the other five fingers;

(h) Applying a protection layer to each nail surface.

Embodiment 2 (nail beautifying painting patterns are created on all ten nails of a pair of hands):

(a) Applying an ink absorbing layer to the surfaces of the ten nails to be beautified, or erasing the grease layer on the nails by using nail oil remover or alcohol;

(b) Selecting eight pictures by using a drawing software in the computer and opening them simultaneously;

(c) Stretching and arranging the eight fingers of both hands except for the two thumbs into a row, and placing them in a finger holder of the painting machine used as the clamp with the figure surfaces facing upward, by means of the finger holder the surfaces of the fingers lined-up side by side are held below the working plane of the painting machine;

(d) Determining the positions of each finger by the camera, and displaying them on the display device of the computer;

(e) Adjusting the position of each picture with respect to its target nail surface by means of operating the computer so that they overlap with each other, and adjusting the size of each picture so that it is slightly larger than the corresponding nail surface, then trimming the edges of each picture by the edge modifying tools of the software so that its edges overlap with the edges of the nail surface;

(f) Issuing the command for printing by means of the computer to the printing device such that the eight pictures are simultaneously printed;

(g) Selecting two pictures by a specific drawing software in the computer and opening them simultaneously;

(h) Arranging the two thumbs side by side, and placing them in a finger holder used as the clamp with the surfaces facing upward, by use of the finger holder the surfaces of the lined-up thumbs are held below the working plane of the painting machine;

(i) Printing patterns on the two thumbs in the same way as the steps (b)-(f);

(j) Applying a protection layer to each nail surface.

The printing sequence of the two group of fingernails (to be printed simultaneously) can be adjusted freely in the above two embodiments.

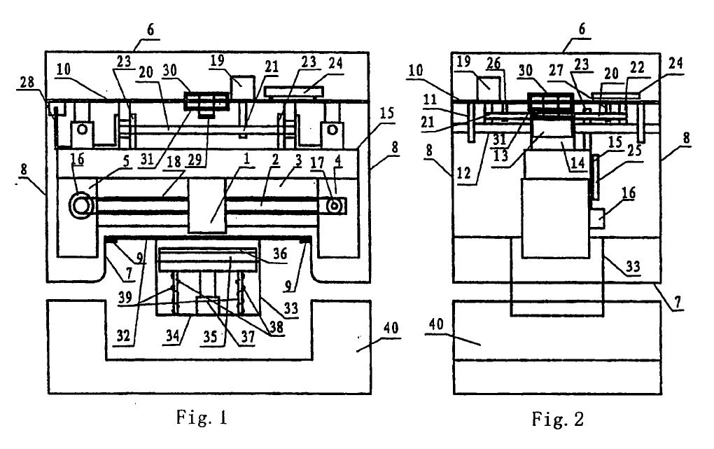

With reference to FIGS. 1-2, the body-painting machine according to the present invention comprises a computer, a printing device, a camera, a longitudinal moving mechanism, a transverse moving mechanism, a clamp and a machine body. The computer is electrically connected to the printing device and the camera. The machine body mainly composed of an upper casing and a base 40, and the upper casing is located above the base. The printing device mainly composed of a printing head 1 which constitutes the moving member of the transverse moving mechanism or is fixedly mounted on the moving member of the transverse moving mechanism. The transverse moving mechanism is constituted of a fixed member, a moving member and a moving member driving means, of which the fixed member is connected to the moving member of the longitudinal moving mechanism. The longitudinal moving mechanism is constituted of a fixed member, a moving member and a moving member driving means, of which the fixed member is connected to the upper part of the inner space of the upper casing. The camera mainly is composed of a camera head 29 which is positioned above the printing head and is arranged facing in the downward direction, whose shooting area covers the printing area of the printing head. There is space provided between the upper casing and the base to receive the clamp, the front side of the space (facing the operator) is open, so that the clamp is movably disposed within the space for receiving the same therein between the upper casing and the base, the position of the body part to be printed determined by which is located within the printing area.

The base 40 can be a case body and is located at the lower portion of the whole device, the upper plate of which presents a U shape when viewed from the front. The mainframe of the computer can be disposed inside the case body of the base. The upper casing can also be a case body, the bottom plate 7 of which assumes a U shape when viewed from the front. There is a hole (square or rectangular hole) formed on the concave surface of the bottom plate, and two longitudinal sliding grooves 9 are formed respectively at upper part of the two side walls of the concave surface. The concave space of the upper plate of the base and the concave space of the bottom plate of the upper casing are interconnected face-to-face so as to form the space for receiving the clamp. The printing device, the camera, the longitudinal moving mechanism, the transverse moving mechanism can all be disposed inside the case body of the upper casing. The printing plane of the printing head is located at the square/rectangular hole of the bottom plate of the upper casing.

The fixed member of the longitudinal moving mechanism mainly comprises a suspending plate 10 and two longitudinal sliding bars 12. The suspending plate is in a planar shape, which is positioned at the upper portion of the inner space of the upper casing and is fastened to the side wall 8 of the upper casing. The two longitudinal sliding bars are parallel to each other which are positioned respectively on the left and right sides below the suspending plate and are fixedly connected to the suspending by their own connecting parts, respectively. The moving member of the longitudinal moving mechanism mainly comprises two linear bearings 13 and a hanger 15. The hanger is located below the two longitudinal sliding bars. The two linear bearings are mounted on the two longitudinal sliding bars respectively, and are fixedly connected to the hanger by their respective fixed brackets 14. The moving member driving means of the longitudinal moving mechanism comprises a step motor 19 and two longitudinal belt transmission means. Each of the two linear bearings is fixedly connected to its corresponding transmission belt 20.1 of the longitudinal belt transmission means. The printing bracket can also be connected to the transmission belts of the two longitudinal belt transmission means respectively by means of a connecting part 23. The step motor is connected to the driving wheels of the two longitudinal belt transmission means by a transverse belt transmission means. The transverse belt transmission means is provided with a driving wheel and two driven wheels. The transverse belt transmission means adopts directly the two driving wheels of the two longitudinal belt transmission means as its two driven wheels. The driving wheel of the transverse belt transmission means is connected to the output shaft of the step motor, and the transmission belt is in T shape with the inner end of its longitudinal portion located at the center of its transverse portion 20.2. Two driven wheels of the transverse belt transmission means are provided at the two ends of the transverse portion of its transmission belt, and a driving wheel 21 is provided at the outer end of the longitudinal portion of said belt. Two driven tightening wheels for tightening the transmission belt are also provided at the outer side of the inner end of the transverse portion of said belt. The connection and installation of each said wheel can be performed with any suitable prior art techniques, such as installed on the suspending plate by means of bearings. The arrangement of this longitudinal moving mechanism can ensure a stable longitudinal movement and synchronous left and right side movements of the moving member driven by the step motor, thereby avoiding the unbalanced phenomenon caused by the one-side transmission under the prior art.

Each of the above-mentioned belt transmission means and those to be used hereinafter can be provided with a regulating wheel 22 for regulating the tension degree of the belt. The regulating wheel can be implemented by any prior art, such as a separately mounted belt pressing wheel or a belt tension wheel, or the tension degree of a belt can also be regulated by using a driven or driving wheel of which the installation position is adjustable.

As for the arrangement of the fixed member and the moving member of the above-mentioned longitudinal moving mechanism, the moving member driving means of said longitudinal moving mechanism can also comprises only a step motor and a longitudinal belt transmission means, and is further provided with a regulating wheel for regulating the degree of tension of the transmission belt and a device for tensing the regulating wheel. Said moving member driving means is positioned below the center portion of the suspending plate and in the co-direction of the center of gravity of both the transverse moving mechanism and the printing device. Its transmission belt being connected to the two linear bearings by the common connection portion and connected to the printing bracket by the connecting part, thereby achieving the synchronous driving of the linear bearings and the printing bracket. The output shaft of the step motor is connected to the driving wheel of the longitudinal moving mechanism. Such longitudinal moving mechanism can also ensure a stable longitudinal movement and synchronous left and right side movements of the moving member driven by the step motor, thereby avoiding the unbalanced phenomenon caused by the one-side transmission under the prior art.

The fixed member of the transverse moving mechanism can mainly comprise the printing bracket 3 and one transverse sliding bar 2. The left and right ends of said printing bracket can be provided with a protecting device and a cleaning device 5 and 4 for the printing head. The transverse sliding bar is positioned below the main portion of the printing bracket with its two ends fixedly connected to the downwardly extended portions of the left and right sides of the printing bracket respectively. The printing head is movably mounted on the transverse sliding bar so as to form the moving member of the transverse moving mechanism. The driving means of the transverse moving mechanism comprises a transverse movement driving motor 16 and a transverse movement belt transmission means. The output shaft of the transverse movement driving motor is fixedly connected to the driving wheel of the transverse movement belt transmission means, and the driving wheel and driven wheel 17 of the transverse movement belt transmission means are mounted to the downwardly extended portions of the left and right sides of the printing bracket. The printing head 1 is fixedly connected to the transmission belt 18 of the transverse movement belt transmission means, thereby achieving left and right movements of the printing head under the driving of the transverse movement driving motor.

The camera head 29 can be mounted on the suspending plate or on the inner side of the upper plate 6 of the upper casing directly or by means of a mounting bracket, and is provided with a light emitter and a control circuit 30 for control of the light emitter. The light emitter and the control circuit for control of the light emitter can be disposed above the camera head, and is mounted on the suspending plate above the working area of the printing head by a regulating board 31. Any prior art can be employed for implementing the light emitter and its control circuit. The light emitted by the light emitter can illuminate the painting location in order to ensure the clearness of the photos taken by the camera.

The clamp can be a hand holder used in nail beautifying, a foot holder used in foot beautifying or a regulating board used in body painting, or any other suitable clamps.

The hand holder can include a hand holder casing consisting of an upper plate 32, side walls 33 and a base 34. The left and right sides of the upper plate of said hand holder can extend outwardly from the connection position thereof with the side walls, so as to form two inserting boards cooperative with the sliding grooves on the bottom plate of the upper casing. The left inserting boards and the right one inserted into their respective sliding grooves, thereby achieving the moveably setting of the hand holder in the space between the upper casing and the base. There is a square/rectangular hole provided at the center of the upper plate of the hand holder, whose position is corresponding to the working plane of the printing head. A slim hole is provided at the engaging position of the front side wall and the upper plate of the hand holder casing so that a plurality of fingers can be placed side by side into it for parallel insertion of a plurality of fingers into the hand holder. Several guiding rods 39 can be distributed on the base of the hand holder casing, on which a spiral spring 38 is disposed. The upper portions of the spiral springs all extend beyond the height of the guiding rods. A support piece is fixedly connected to the upper end of each spiral spring and the support piece of each spiral spring can be a whole piece 35 with a piece of elastic sponge 36 laid on it. When in use, all the fingers for painting are placed simultaneously onto the sponge piece. However, the support piece on each spiral spring can also be independent pieces different from each other with or without a piece of sponge laid on it. When in use, different fingers can be placed on different support pieces or the sponge pieces of different support pieces. A plurality of locking switch devices 37 can further be provided on the base of the hand holder, each of which is connected to a corresponding support piece for locking the support piece at or releasing it from the pressed-down position so that the support piece is stretched upward by means of the spiral spring, resulting in the fingers placed on the elastic sponge piece or the support pieces being positioned right in the printing area of the printing head. The elasticity of the elastic sponge piece and the spiral spring can ensure that the fingers be contacted with the upper plate of the hand holder after the support pieces went upward so that the fingers are located at a proper height. The locking switch device can be constructed with any suitable prior art, such as an elastic pin structure.

The control circuit of the device according to the present invention consists of two control circuit boards 24 and 25, two signaling boards installed at different positions, and a switch indicator circuit installed at the front side of the outer casing, which are connected respectively with each of the motors and the computer. The signaling board can be constructed with two limit switch buttons 26, 27 and a trigger 28, or with two photoelectric sensors and a light-shielding sheet for sensing the position signals and position controlling. The structures, the installing positions and installing methods of these circuit boards, signaling board, and the switch indicator circuit, and the connection with each of the motors and the computer and the control modes can be set up with the prior art. The structures, the interconnections and the control modes of the computer, the printing head and the camera head can also be set up with the prior art.

The computer in the present invention can be a personal computer of any type. Various kinds of pictures can be pre-stored in the computer. the program for picture processing and other operating can be developed in any prior art software tools such as Delphi, Vc++, etc. The required various operations including picture edge trimming can be achieved with the prior art. The various medicaments such as base oil, protection layer coating materials, and easy-washed coatings, etc. required in body painting can be those used in the prior art for nail beautifying and body beautifying.

The method and device according to the present invention can be used in multi-patterns body painting, and can -also be used in multi-patterns painting of other objects. Therefore the latter realized with the method and device of the present invention should be regarded as the embodiments of the present invention.

What is claimed is:

1. A body painting method for simultaneously creating a plurality of patterns, which employs a specialized computer-controlled body-painting machine to print the body-painting patterns, comprising the following basic steps:
(1) Opening a plurality of pictures used for body paintings simultaneously in a computer;
(2) Placing objects having a plurality of parts to be painted onto a corresponding clamp, and holding each part within the printing area of a printing device by means of the clamp;
(3) Determining the location of each part by a camera and displaying them on a display of the computer, and adjusting the size and printing location of each opened picture through the computer so that the size of each picture is equal to or larger than the size of its respective body part to be painted while the printing location of each picture is positioned at said respective body part;
(4) Starting the printing device by the computer so that all pictures are printed as one pattern by the printing device, while each picture is printed right onto its respective body part.

2. The body painting method for simultaneously creating a plurality of patterns according to claim 1, characterized in that a preprocess for the body parts to be painted can be performed according to the properties of different body parts so as to meet the requirements of printing and human body protection; and a post-process can also be performed upon completion of printing so as to meet the requirements of aesthetics and protection of the printed patterns.

3. The body painting method for simultaneously creating a plurality of patterns according to claim 2, characterized in that said step (3) includes following steps: taking a static photo illustrating the location of each body part to be printed by the camera; displaying these static photos on the bottom layer of the adjusting working area for the opened pictures of the display of the computer; adjusting each of the opened pictures on the display to respective position which corresponds to painting body part; presenting each picture on the display semi-transparently so that the profile of the nail beneath the picture can be seen clearly; adjusting the size of each picture so that it is slightly larger than that of the painting body part; and then drawing a profile line of the painting location; removing the pixels of the picture beyond the profile so that the edges of the picture overlap the edges of the nail.

4. The body painting method for simultaneously creating a plurality of patterns according to claim 2, characterized in that said preprocess further includes the step of applying a coating that can be readily washed off to the surrounding of the painting body part; and the size of each picture is adjusted in step (3) so that it is slightly larger than that of the painting body part, and those parts of the body-painting pattern outside the painting body part are then erased using suitable detergents after printing.

5. The body painting method for simultaneously creating a plurality of patterns according to claims 3 or 4, characterized in that the fingernails can be divided into two groups according to the following two modes during the painting process, and all the fingernails are preprocessed before printing and are post-processed after printing:
Mode 1: All the four fingers except for the thumb of one hand and the thumb of the other hand are placed in one group, and the other five fingers are placed in another group;
Mode 2: The eight fingers of both hands except for the two thumbs are placed together to be a group, and the two thumbs are to be another group;

6. A computer-controlled body-painting machine for simultaneously creating a plurality of patterns, characterized in that the machine comprises a computer, a printing device, a camera, a longitudinal moving mechanism, a transverse moving mechanism, a clamp and a machine body, wherein said computer is electrically connected to the printing device and the camera, the machine body is mainly composed of an upper casing and a base, the upper casing locates above the base, the printing device mainly comprises a printing head which forms a moving member of the transverse moving mechanism or is fixedly mounted on a moving member of the transverse moving mechanism, the transverse move mechanism comprises a fixed member, a moving member and a moving member driving means, the fixed member of which is connected to a moving member of the longitudinal moving mechanism, the longitudinal moving mechanism comprises a fixed member, a moving member and a moving member driving means, the fixed member of which is connected to the upper part of the inner space of the upper casing, the camera mainly comprises a camera head which is positioned above the printing head, and is arranged in such way that the shooting direction thereof facing downward and the shooting area covers the printing area of the printing head, there is provided with a space between the upper casing and the base to receive the clamp, the front side of the space is opened, the clamp is movably disposed within the space for receiving the same therein between the upper casing and the base, the position of the painting body part determined by which is located within the printing area.

7. The computer-controlled body-painting machine for simultaneously creating a plurality of patterns according to claim 6, characterized in that the base is a case body and is positioned at the lower portion of the whole device, whose upper plate assumes a U shape when viewing from the front, the mainframe of the computer is disposed inside the case body of the base; the upper casing is a case body, whose bottom plate assumes a invert U shape when viewing from the front; there is a square hole formed on the concave surface of the bottom plate, two longitudinal sliding grooves are formed respectively at the upper parts of the two side walls of the concave of the bottom plate, the concave space of the upper plate of the base and the concave space of the bottom plate of the upper casing are interconnected face-to-face so as to form the space for receiving the clamp; the printing device, the camera, the longitudinal moving mechanism, the transverse moving mechanism are all disposed inside the case body of the upper casing, and the printing plane of the printing head is positioned at the square hole of the bottom plate of the upper casing; the fixed member of the transverse moving mechanism can mainly comprise a printing bracket and one transverse sliding bar, the left and right ends of said printing bracket is provided with a protecting and cleaning device for the printing head, the transverse sliding bar is positioned below the main portion of the printing bracket with its two ends fixedly connected to the downwardly extended portions of the left and right sides of the printing bracket respectively, and the printing head is movably mounted on the transverse sliding bar thereby forming the moving member of the transverse moving mechanism; the driving means of the transverse moving mechanism comprises a transverse movement driving motor and a transverse movement belt transmission means, the output shaft of the transverse movement driving motor is fixedly connected to the driving wheel of the belt transmission means, and the driving wheel and driven wheel of the transverse movement belt transmission means are mounted to the downwardly extended portions of the left and right sides of the printing bracket respectively, the printing head is fixedly connected to the transmission belt of the transverse movement belt transmission means; the camera head is mounted on the suspending plate or on the inner side of the upper plate of the upper casing directly or by means of a mounting bracket, and is provided with a light emitter and a control circuit for control of the light emitter, the light emitter and the control circuit for control of the light emitter is disposed above the camera head, and is mounted on the suspending plate above the working area of the printing head by a regulating board.

8. The computer-controlled body-painting machine for simultaneously creating a plurality of patterns according to claim 7, characterized in that the fixed member of the longitudinal moving mechanism mainly comprises a suspending plate and two longitudinal sliding bars, the suspending plate is in a planar shape, which is positioned at the upper portion of the inner space of the upper casing and is secured to the side wall of the upper casing; the two longitudinal sliding bars are parallel to each other, which are positioned respectively on the left and right sides below the suspending plate and are fixedly connected to the suspending plate by their respective connecting parts; the moving member of the longitudinal moving mechanism mainly comprises two linear bearings and a hanger, the hanger is located below the two longitudinal sliding bars and the two linear bearings are movably mounted on the two longitudinal sliding bars respectively and are fixedly connected to the hanger by their respective fixed brackets; the moving member driving means of the longitudinal moving mechanism comprises a step motor and two longitudinal belt transmission means, each of the two linear bearings is fixedly connected to its corresponding transmission belt of the longitudinal belt transmission means, the step motor is connected to the driving wheels of the two longitudinal belt transmission means by a transverse belt transmission means, the transverse belt transmission means is provided with a driving wheel and two driven wheels, the two driven wheels of the transverse belt transmission means may be respectively coaxial with the driving wheels of the two longitudinal belt transmission means or directly employ the driving wheels of the two longitudinal belt transmission means, the driving wheel of the transverse belt transmission means is connected to the output shaft of the step motor, and the transmission belt is in T shape with the inner end of its longitudinal portion located at the center of its transverse portion, two driven wheels of the transverse belt transmission means are provided at the two ends of the transverse portion of its transmission belt, a driving wheel-is provided at the outer end of the longitudinal portion of said transmission belt and two driven tightening wheels for tightening the transmission belt are also provided at the outer side of the inner end of the transverse portion of said belt; the moving member of the transverse moving mechanism, the driving means of the transverse moving mechanism comprises a transverse movement driving motor and a transverse movement belt transmission means, the output shaft of the transverse movement driving motor is fixedly connected to the driving wheel of the transverse movement belt transmission means, and the driving wheel and driven wheel of the transverse movement belt transmission means are mounted to the downwardly extended portions of the left and right sides of the printing bracket, the printing head is fixedly connected to the transmission belt of the transverse movement belt transmission means.

9. The computer-controlled body-painting machine for simultaneously creating a plurality of patterns according to claim 7, characterized in that the fixed member of the longitudinal moving mechanism mainly comprises a suspending plate and two longitudinal sliding bars, the suspending plate is in a planar shape, which is positioned at the upper portion of the inner space of the upper casing and is secured to the side wall of the upper casing, the two longitudinal sliding bars are parallel to each other which are located respectively on the left and right sides below the suspending plate and are fixedly connected to the suspending plate by their respective connecting parts; the moving member of the longitudinal moving mechanism mainly comprises two linear bearings and a hanger, the hanger is located below the two longitudinal sliding bars and the two linear bearings are movably mounted on the two longitudinal sliding bars respectively and are fixedly connected to the hanger by their respective fixed brackets, the moving member driving means of the longitudinal moving mechanism can also comprises only a step motor and a longitudinal belt transmission means, and is further provided with a regulating wheel for regulating the degree of tension of the transmission belt and a device for tensing the regulating wheel, said moving member driving means is positioned below the center portion of the suspending plate and in the co-direction of the centers of gravity of both the transverse moving mechanism and the printing device, and its transmission belt being connected to the two linear bearings by the common connection portion and connected to the printing bracket by the connecting part; the output shaft of the step motor is connected to the driving wheel of the longitudinal moving mechanism; the moving member of the transverse moving mechanism, the driving means of the transverse moving mechanism comprises a transverse movement driving motor and a transverse movement belt transmission means, the output shaft of the transverse movement driving motor is fixedly connected to the driving wheel of the transverse movement belt transmission means, and the driving wheel and driven wheel of the transverse movement belt transmission means are mounted to the downwardly extended portions of the left and right sides of the printing bracket, the printing head is fixedly connected to the transmission belt of the transverse movement belt transmission means.

10. The computer-controlled body-painting machine for simultaneously creating a plurality of patterns according to claims 6, 7, 8, or 9, characterized in that the clamp is a hand holder, the hand holder includes a hand holder casing consisting of an upper plate, side walls and a base; the left and right sides of the upper plate of said hand holder extend outwardly from the connection position between the upper plate with the side walls so as to form two inserting boards cooperative with the sliding grooves on the bottom plate of the upper casing; said left and right inserting boards inserts into the respective sliding grooves; there is a square hole provided at the center of the upper plate of the hand holder, the position of the square hole is corresponding to the working plane of the printing head; a slim hole is provided at the engaging position of the front side wall of the hand holder casing and the upper plate of the hand holder; several guiding rods are distributed on the base of the hand holder casing, each of which is inserted into a spiral spring; the upper portion of each one of the spiral springs extends beyond the height of each one of the guiding rods; a support piece is fixedly connected to the upper end of each spiral spring and the support piece of each spiral spring can be a whole piece with a piece of elastic sponge laid on it or different supporting pieces independent from each other; a plurality of locking switch devices are further provided on the base of the hand holder, each of which is connected to a corresponding support piece and used for locking the support pieces at suppressing position or releasing them.
